(19) United States
(12) Patent Application Publication
(10) Pub. No.: US 2010/0004449 A1
(43) Pub. Date: Jan. 7, 2010

(54) CRYSTALLINE FORMS OF ERLOTINIB BASE AND ERLOTINIB HCL

(75) Inventors: Ales GAVENDA, Ostrava-Lhotka (CZ); Pavel VRASPIR, Rymarov (CZ); Augusto CANAVESI, Locate Varesino(CO) (IT); Judith ARONHIME, Rehovot (IL); Ettore BIGATTI, Rho (IT); Jiri FAUSTMANN, Opava-6 (CZ); Alexandr JEGOROV, Dobra Voda (CZ); Peter W. STEPHENS, Stony Brook, NY (US); Giovanna LUX, Milano (IT); Maurizio PAIOCCHI, Milano (IT)

Correspondence Address: MERCHANT & GOULD PC, P.O. BOX 2903, MINNEAPOLIS, MN, 55402-0903, US

(73) Assignee: PLUS CHEMICALS SA, Paradiso (CH)

(21) Appl. No.: 12/498,543

(22) Filed: Jul. 7, 2009

Related U.S. Application Data
(60) Provisional application No. 61/078,694, filed on Jul. 7, 2008.
(60) Provisional application No. 61/079,725, filed on Jul. 10, 2008.
(60) Provisional application No. 61/084,553, filed on Jul. 29, 2008.
(60) Provisional application No. 61/084,789, filed on Jul. 30, 2008.
(60) Provisional application No. 61/085,227, filed on Jul. 31, 2008.
(60) Provisional application No. 61/086,032, filed on Aug. 4, 2008.
(60) Provisional application No. 61/086,616, filed on Aug. 6, 2008.
(60) Provisional application No. 61/108,735, filed on Oct. 27, 2008.
(60) Provisional application No. 61/117,729, filed on Nov. 25, 2008.
(60) Provisional application No. 61/149,550, filed on Feb. 3, 2009.

(51) Int. Cl. C07D 239/72

(52) U.S. Cl. 544/293

(57) ABSTRACT

The preparation of crystalline Erlotinib base form G2 is described. This crystalline form can be converted to an Erlotinib salt, such as Erlotinib HCl, which can be used in the treatment of patients with locally advanced or metastatic non-small cell lung cancer (NSCLC).

### CROSS-REFERENCE TO RELATED APPLICATIONS

This application claims the benefit of U.S. Provisional Patent Application Ser. Nos. 61/078,694, filed Jul. 7, 2008; 61/079,725, filed Jul. 10, 2008; 61/084,553, filed Jul. 29, 2008; 61/084,789, filed Jul. 30, 2008; 61/085,227, filed Jul. 31, 2008; 61/086,032, filed Aug. 4, 2008; 61/086,616, filed Aug. 6, 2008; 61/108,735, filed Oct. 27, 2008; 61/117,729, filed Nov. 25, 2008; 61/149,550, filed Feb. 3, 2009, which are incorporated herein by reference.

### FIELD OF THE INVENTION

The present invention relates to a process to prepare crystalline form G2 of Erlotinib base, a process to prepare a crystalline form of Erlotinib HCl characterized by data selected from the group consisting of: a powder XRD pattern having peaks at about 10.1 and 17.4±0.2 degrees 2-theta and any 3 peaks selected from the list consisting of: 5.7, 10.1, 17.4, 18.9, 21.3, 23.6 and 29.3±0.2 degrees 2-theta, a PXRD pattern described in FIG. 4, and combinations thereof and to crystalline form AL of Erlotinib HCl.

### BACKGROUND OF THE INVENTION

Erlotinib HCl, N-(3-ethynylphenyl)-6,7-bis(2-methoxyethoxy)-4-quinazolinamine hydrochloride, of the following formula

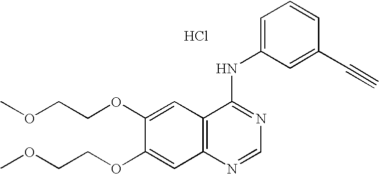

is marketed under the trade name TARCEVA® by OSI Pharmaceuticals for treatment of patients with locally advanced or metastatic non-small cell lung cancer (NSCLC) after failure of at least one prior chemotherapy regimen.

Erlotinib (ERL) and its preparation are disclosed in U.S. Pat. No. 5,747,498, where the free base is produced, as shown in Scheme 1

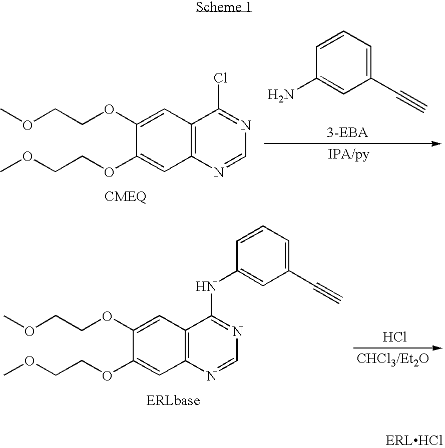

In this process, the reaction of 3-ethynylaniline (3-EBA) with 4-chloro-6,7-bis(2-methoxyethoxy)quinazoline (CMEQ) in a mixture of pyridine and isopropanol (IPA) yields the free base, which is purified by chromatography on silica gel using a mixture of acetone and hexane. The free base is then converted into the hydrochloride salt by treating a solution of ERL base in CHCl₃/Et₂O with HCl.

U.S. Pat. No. 6,900,221 discloses Form A that exhibits an X-ray powder diffraction pattern having characteristic peaks expressed in degrees 2-theta at approximately 5.579, 9.84, 11.25, 18.86, 19.517, 22.70, 23.50, 24.18, 24.59, 25.40, and 29.24; and Form B substantially free of Form A, wherein Form B exhibits an X-ray powder diffraction pattern having characteristics peaks expressed in degrees 2-theta at approximately 6.26, 12.48, 13.39, 16.96, 20.20, 21.10, 22.98, 24.46, 25.14, and 26.91.

U.S. Pat. No. 6,900,221 also states that “the hydrochloride compound disclosed in U.S. Pat. No. 5,574,498 actually comprised a mixture of the polymorphs A and B, which because of its partially reduced stability (i.e., from the polymorph A component) was not more preferred for tablet form than the mesylate forms.”

This patent also reports that the use of IPA as a solvent for preparing Form A is not recommended due to the formation of an impurity by reaction of the solvent with CMEQ.

U.S. Pat. No. 6,476,040 discloses methods for the production of ERL and salts thereof by treatment of 4-[3-[[6,7-bis(2-methoxyethoxy]-4-quinazolinyl]amino]phenyl]-2-methyl-3-butyn-2-ol with sodium hydroxide and then with HCl in IPA, 2-methoxyethanol, 2-butanol and n-butanol) as reported in Scheme 2.

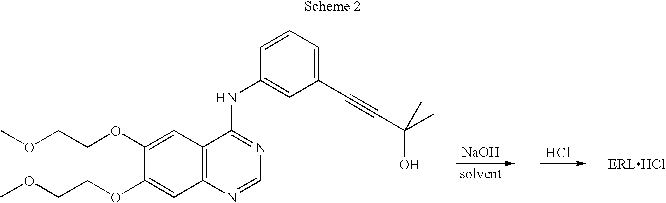

U.S. Pat. No. 7,148,231 discloses Forms A, B, E, which are characterized by X-Ray powder diffraction, IR and melting point.

The isolation of erlotinib is also disclosed in P. Knesl, et al., “Improved Synthesis of Substituted 6,7-Dihydroxy-4-quinazolineamines: Tandutinib, Erlotinib and Gefitinib,” Molecules 11: 286-297 (2006) (“Knesl article”). The Knesl article reports the isolation of erlotinib by extracting with dichlorormethane (DCM) a solution of erlotinib hydrochloride after basification with concentrated ammonia, followed by evaporating the solvent to obtain a product having a melting point of 159 to 160° C.

U.S. Patent Application Publication No. 20090012295 discloses polymorphs G1, G2, G3, and amorphous of erlotinib base, and processes for the preparation thereof. Crystalline erlotinib form G2 is characterized by data selected from the group consisting of: an X-ray powder diffraction pattern with peaks at about 6.5, 12.9, 17.3, 18.3 and 22.4 degrees two-theta±0.2 degrees two-theta, and a PXRD pattern as depicted in FIG. 9.

The present invention addresses the need for additional processes to prepare crystalline Erlotinib base form G2 as well as other processes to prepare crystalline Erlotinib HCl.

The present invention also relates to the solid state physical properties of Erlotinib HCl. These properties can be influenced by controlling the conditions under which Erlotinib HCl is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take this fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance that can be identified unequivocally by X-ray spectroscopy. The polymorphic form may give rise to thermal behavior different from that of the amorphous material or another polymorphic form. Thermal behavior is measured in the laboratory by such techniques as capillary melting point, thermo gravimetric analysis (TGA) and differential scanning calorimetry (DSC) and can be used to distinguish some polymorphic forms from others. A particular polymorphic form may also give rise to distinct spectroscopic properties that may be detectable by solid state 13C NMR spectrometry and infrared spectroscopy.

One of the most important physical properties of a pharmaceutical compound, which can form polymorphs or solvates, is its solubility in aqueous solution, particularly the solubility in gastric juices of a patient. Other important properties relate to the ease of processing the form into pharmaceutical dosages, as the tendency of a powdered or granulated form to flow and the surface properties that determine whether crystals of the form will adhere to each other when compacted into a tablet.

The discovery of new polymorphic forms of a pharmaceutically useful compound such as Erlotinib HCl provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. Thus, there is a need for new polymorphs of erlotinib HCl.

### SUMMARY OF THE INVENTION

In one embodiment, the present invention encompasses a process for preparing crystalline form of Erlotinib base characterized by data selected from the group consisting of: an X-ray powder diffraction pattern with peaks at about 6.5, 12.9, 17.3, 18.3 and 22.4 degrees two-theta±0.2 degrees two-theta, and a PXRD pattern as depicted in FIG. 7 (Form G2), which comprises reacting sodium acetate and erlotinib hydrochloride in an alcohol to obtain a suspension containing crystalline Erlotinib base form G2.

In yet another embodiment, the present invention encompasses processes for preparing Erlotinib salt comprising preparing Erlotinib base form G2, according to the procedure described herein and converting it to Erlotinib salt. Preferably, the Erlotinib salt is Erlotinib HCl.

### BRIEF DESCRIPTION OF THE FIGURES

FIG. 1 illustrates a PXRD pattern of crystalline Erlotinib hydrochloride designated Form AL.

FIG. 2 illustrates a zoomed PXRD pattern of crystalline Erlotinib hydrochloride designated Form AL.

FIG. 3 illustrates a zoomed calculated PXRD pattern from structure determination data (at 25° C.) of crystalline Erlotinib hydrochloride designated Form AL.

FIG. 4 illustrates the PXRD pattern of crystalline Erlotinib hydrochloride characterized by data selected from the group consisting of: a powder XRD pattern having peaks at about 10.1 and 17.4±0.2 degrees 2-theta and any 3 peaks selected from the list consisting of: 5.7, 10.1, 17.4, 18.9, 21.3, 23.6 and 29.3±0.2 degrees 2-theta, a PXRD pattern described in FIG. 4, and combinations thereof.

FIG. 5 illustrates the DSC thermogram of crystalline Erlotinib hydrochloride characterized by data selected from the group consisting of: a powder XRD pattern having peaks at about 10.1 and 17.4±0.2 degrees 2-theta and any 3 peaks selected from the list consisting of: 5.7, 10.1, 17.4, 18.9, 21.3, 23.6 and 29.3±0.2 degrees 2-theta, a PXRD pattern described in FIG. 4, and combinations thereof.

FIG. 6 illustrates the microscope image of crystalline Erlotinib hydrochloride characterized by data selected from the group consisting of: a powder XRD pattern having peaks at about 10.1 and 17.4±0.2 degrees 2-theta and any 3 peaks selected from the list consisting of: 5.7, 10.1, 17.4, 18.9, 21.3, 23.6 and 29.3±0.2 degrees 2-theta, a PXRD pattern described in FIG. 4, and combinations thereof.

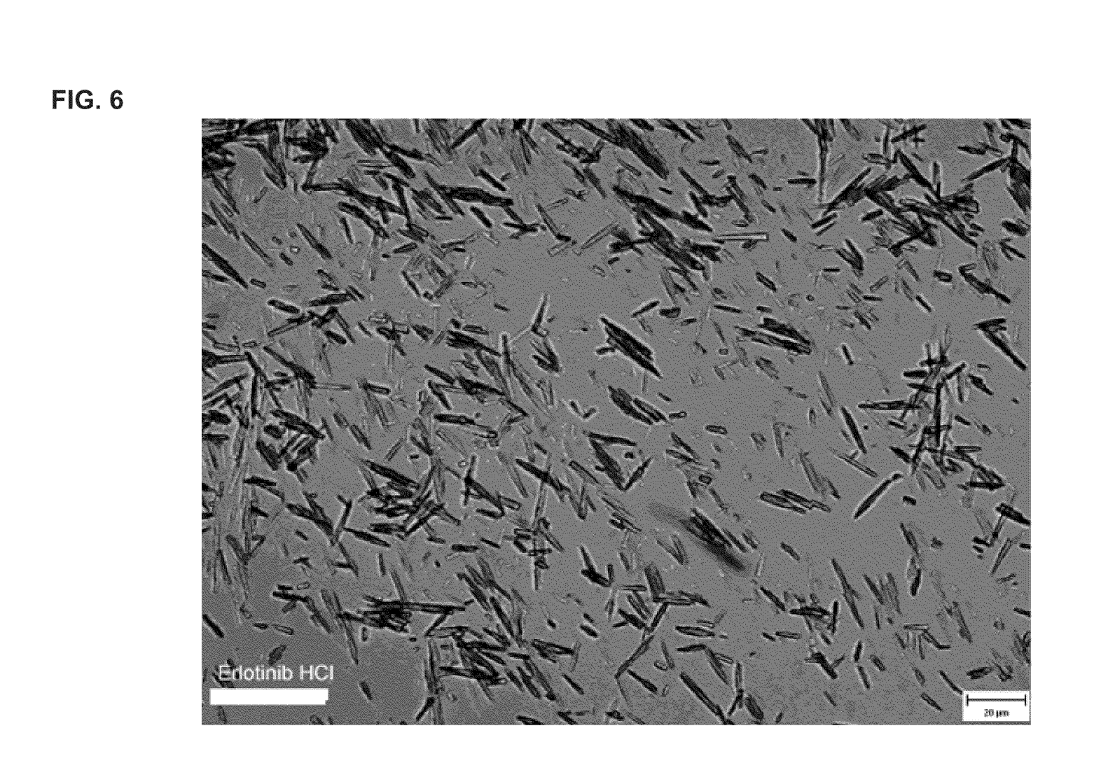

FIG. 7 illustrates an X-ray powder diffraction pattern of crystalline form G2 of Erlotinib base.

FIG. 8 shows an X-ray powder diffraction pattern of crystalline erlotinib base Form G2 containing NaCl (diffractions of NaCl are marked by * in the diffraction pattern).

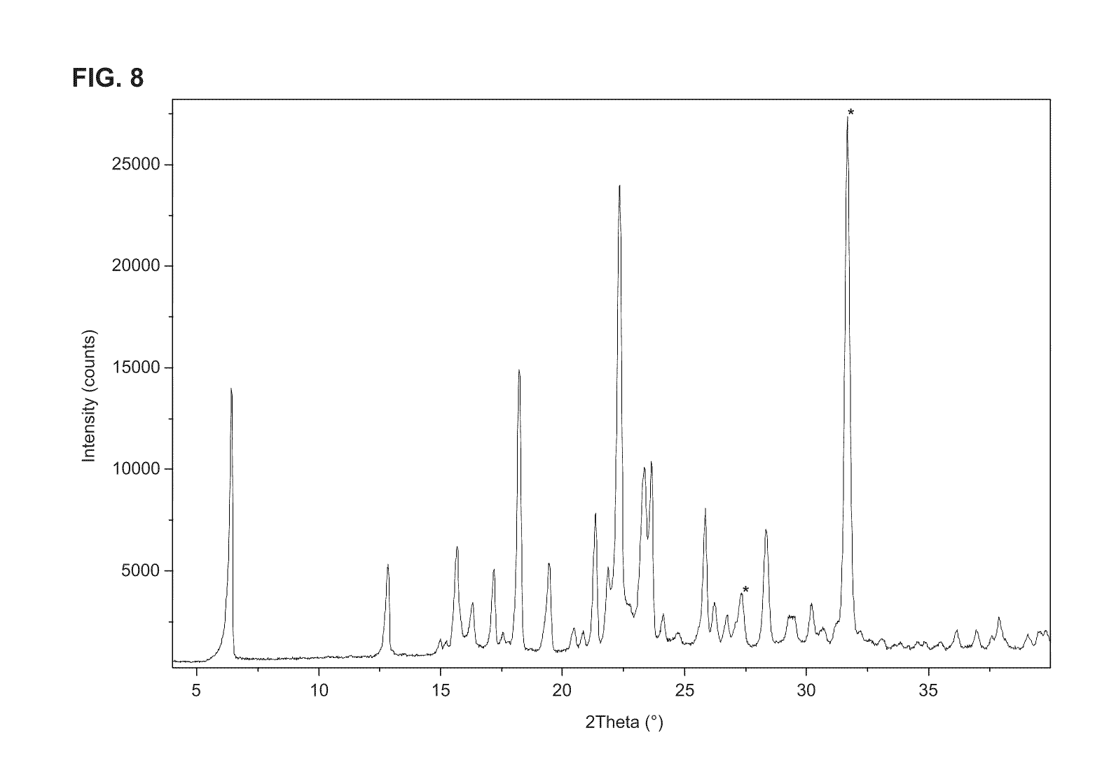

### DETAILED DESCRIPTION OF THE INVENTION

The present invention relates to a process to prepare crystalline form G2 of Erlotinib base, a process to prepare a crystalline form of Erlotinib HCl and to crystalline form AL of Erlotinib HCl.

In one embodiment, the present invention is directed to process for the preparation of crystalline erlotinib base form G2.

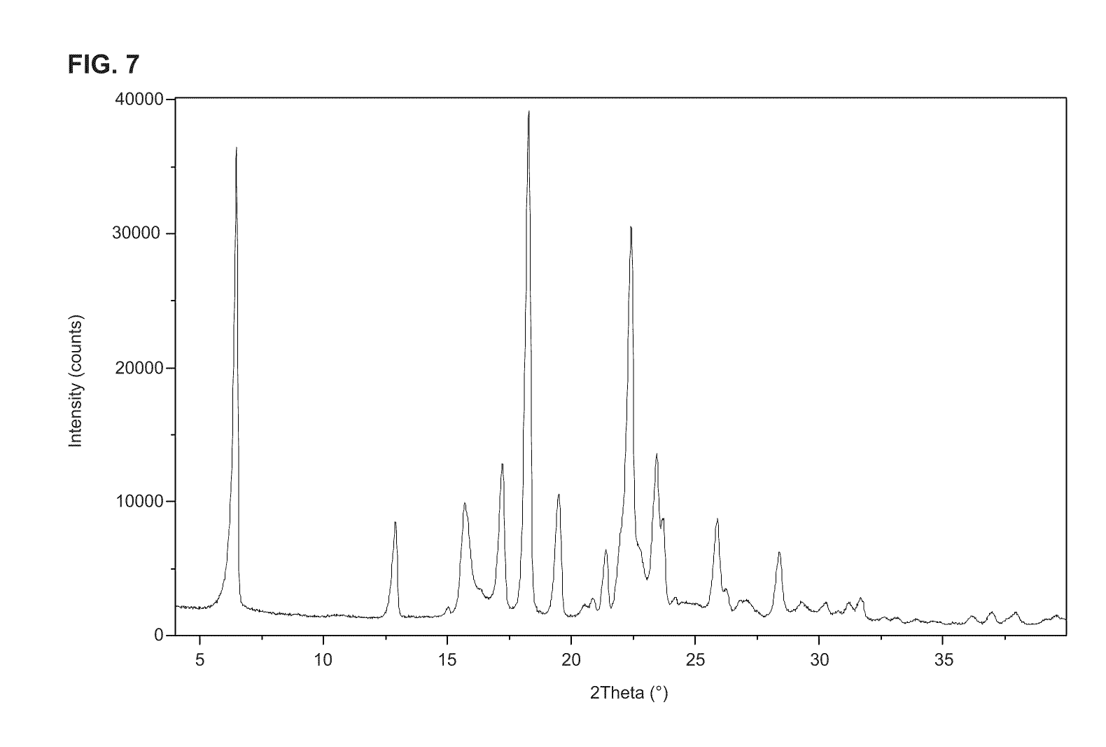

As used herein, the term “crystalline Erlotinib base form G2” refers to crystalline Erlotinib base characterized by data selected from the group consisting of: an X-ray powder diffraction pattern with peaks at about 6.5, 12.9, 17.3, 18.3 and 22.4 degrees two-theta±0.2 degrees two-theta, and a PXRD pattern as depicted in FIG. 7.

The process comprises reacting sodium acetate and erlotinib hydrochloride in an alcohol, to obtain a suspension containing crystalline Erlotinib base form G2.

The starting erlotinib hydrochloride may be obtained, for example, according to the process described in Example 3.

The starting Erlotinib HCl can be neat (i. e., without a solvent) or in a reaction mixture where it is formed. Typically, the reaction mixture may comprise a solvent, e.g., an alcohol, preferably, C₁₋₄ alcohol, more preferably, C₁₋₃ alcohol, most preferably, isopropanol.

In one embodiment, the sodium acetate may be added to the reaction mixture comprising erlotinib hydrochloride and the alcohol to obtain a suspension comprising the said crystalline form of Erlotinib base. The addition of sodium acetate neutralizes erlotinib hydrochloride to form erlotinib base form G2 and sodium chloride, which precipitate.

When the starting Erlotinib HCl is in a reaction mixture where it is formed, this reaction mixture can be a reaction mixture heated to an elevated temperature, such as about 30° C. to about reflux temperature, preferably, to about 35° C. to about 50° C., most preferably to about 40° C. If the reaction mixture is at an elevated temperature, it is preferably cooled prior to the reaction with of sodium acetate. Preferably, cooling may be done to a temperature of about 15° C. to about 30° C., more preferably, 20° C. to about 30° C., most preferably, to about 20° C. to about 25° C.

Optionally, the precipitate is then recovered from the suspension. The recovery can be done, for example, by filtering the suspension, washing the filtered precipitate, and drying. Preferably, drying is performed at a temperature range of about 40° C. to about 60° C., more preferably, of about 50° C. Preferably, drying time may be for at least about 2 hours to about 8 hours, more preferably, 3 hours to about 6 hours, most preferably, for about 3 hours.

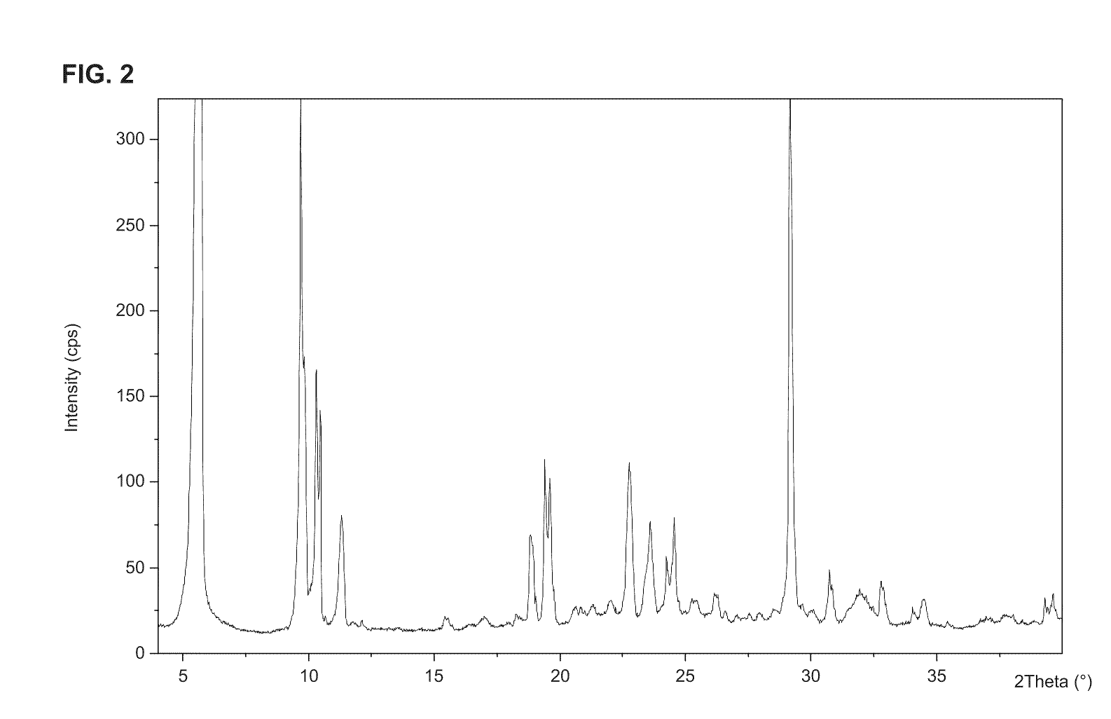

The recovered precipitate can contain traces of NaCl that can be identified in the pattern depicted in FIG. 2, by the peaks at 27.3 and 31.7±degrees two-theta±0.2 degrees two-theta.

The separation of Erlotinib base form G2 from NaCl and its conversion to Erlotinib salt can be achieved by suspending the precipitate in a water-immiscible organic solvent, preferably, a water-immiscible ketone, most preferably, methyisobutylketone (“MIBK”) and water, thereby producing a mixture. The mixture is stirred under heating, for example to a temperature of about 65° C. to about 70° C., until the phases are separated. The aqueous phase containing the salts (e.g. NaCl) is removed, and the organic phase containing erlotinib base is acidified to give the corresponding acid salt. Preferably, the salt is HCl.

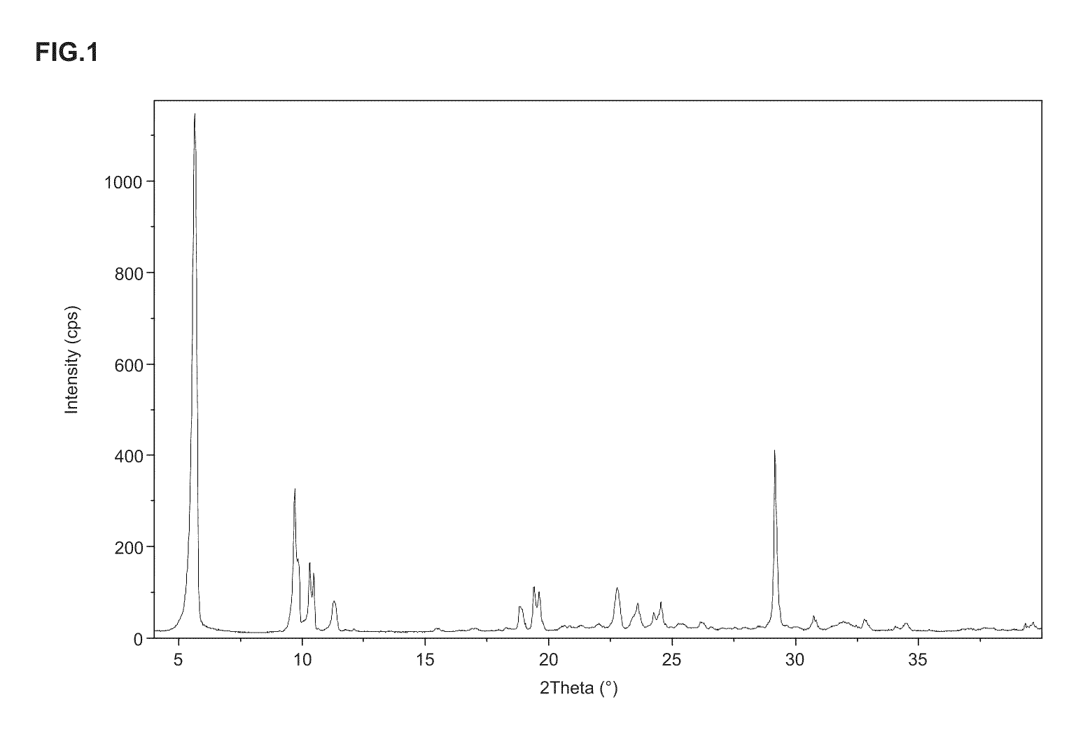

The present invention also encompasses crystalline Erlotinib HCl, designated form AL, characterized by data selected from the group consisting of: a powder XRD pattern having peaks at about 10.5 and 22.1±0.2 degrees two-theta, and any 3 peaks selected from the list consisting of 5.7, 9.8, 11.4, 13.2, 13.6, 16.5, 18.1 and 20.7±0.2 degrees 2-theta, and also does not contain diffraction peaks at 10.1 and 17.4±0.2 degrees; a PXRD pattern depicted in FIG. 1; a PXRD pattern depicted in FIG. 2, and combination thereof.

The above crystalline form AL can be prepared by a process comprising crystallizing Erlotinib HCl from methylethylketone (“MEK”).

The starting erlotinib base can be prepared for example, by the process disclosed in U.S. Pat. No. 5,747,498.

The crystallization preferably comprises providing a solution of Erlotinib HCl in MEK and precipitating the said crystalline form to obtain a suspension.

Preferably, the solution is prepared by dissolving erlotinib base in MEK and reacting the said solution with HCl.

Dissolution of Erlotinib base in MEK can be achieved by heating a mixture comprising Erlotinib base and MEK. Preferably, heating is to about 50° C. to about 70° C. Typically, the heated solution is cooled prior to the reaction with HCl. Preferably, it is cooled to a temperature of about 15° C. to about 25° C., more preferably, to about 20° C.

Said precipitation is achieved as soon as the solution containing Erlotinib base reacts with HCl. Preferably, vapors of HCl react with the solution of erlotinib base. The vapors are formed by adding an aqueous solution of HCl to a closed vessel, wherein this closed vessel also contains the solution of erlotinib base. Preferably, the addition is done by dripping the HCl solution to the bottom of the closed vessel.

Preferably, HCl diffusion is done for about 3 days, wherein during this time the reaction between erlotinib base and the HCl vapors takes place.

Preferably, concentration of said aqueous solution of HCl is about 30% to about 50% by weight, more preferably, of about 35% to about 44.1% by weight.

The process for preparing crystalline form AL may further comprise recovery of the said crystalline form. Preferably, the said recovery comprises:

a) separation of the precipitated crystalline Erlotinib-HCl from the mother liquor,

b) washing, and

c) drying the separated crystalline form.

Preferably, the crystalline form is separated by filtration. Preferably, washing is done with t-butyl methyl ether (“TBME”). Preferably, drying is done by air.

Isolation and single-crystal XRD analysis of one crystal from this sample provides the following structure, where the unit cell parameters approximately equal to the following:

### Cell Dimensions (Measured at Temperature 200 K):

| | |
|---|---|
| cell_length_a | 18.232(3) Å |
| cell_length_b | 7.4474(13) Å |
| cell_length_c | 33.421(5) Å |
| cell_angle_alpha | 90 deg. |
| cell_angle_beta | 111.860(18) deg. |
| cell_angle_gamma | 90 deg. |
| cell_volume | 4211.6(13) Å |
| symmetry_cell_setting | ‘Monoclinic’ |
| symmetry_space_group_name_H-M | P21/c (No. 14) |

### Cell Dimensions (Calculated at Temperature 25° C.):

| | |
|---|---|
| cell_length_a | 18.27 Å |
| cell_length_b | 7.52 Å |
| cell_length_c | 33.59 Å |
| cell_angle_alpha | 90 deg. |
| cell_angle_beta | 112.2 deg. |
| cell_angle_gamma | 90 deg. |
| symmetry_cell_setting | ‘Monoclinic’ |
| symmetry_space_group_name_H-M | P21/c (No. 14) |

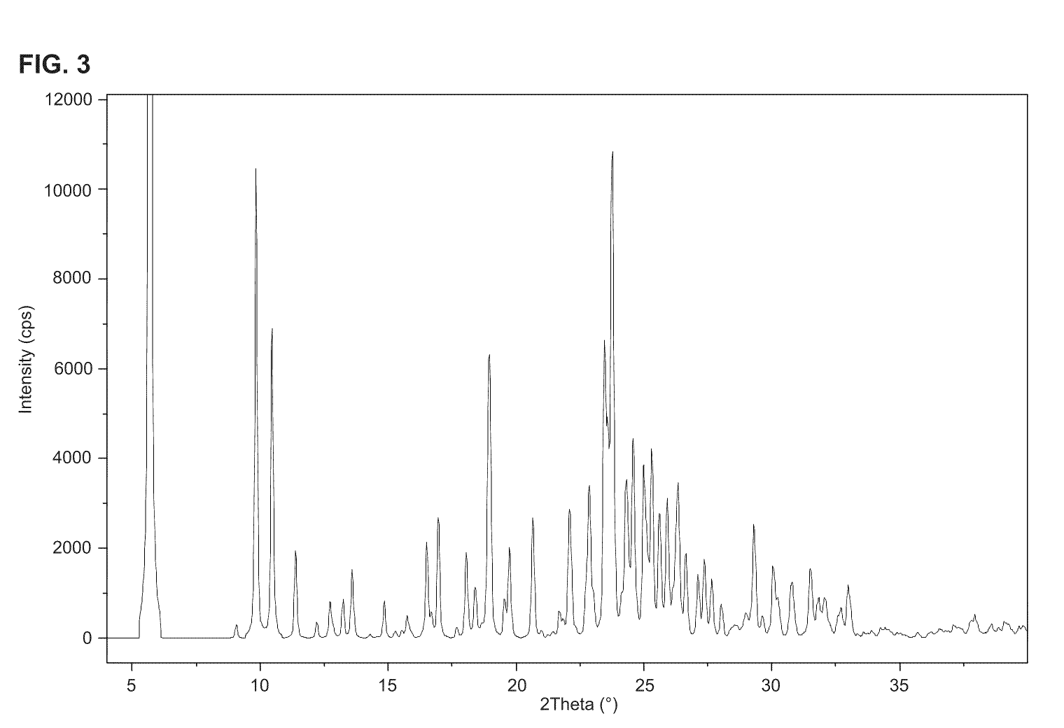

The calculated PXRD pattern from the single crystal structure (at 25° C.) is shown in FIG. 3.

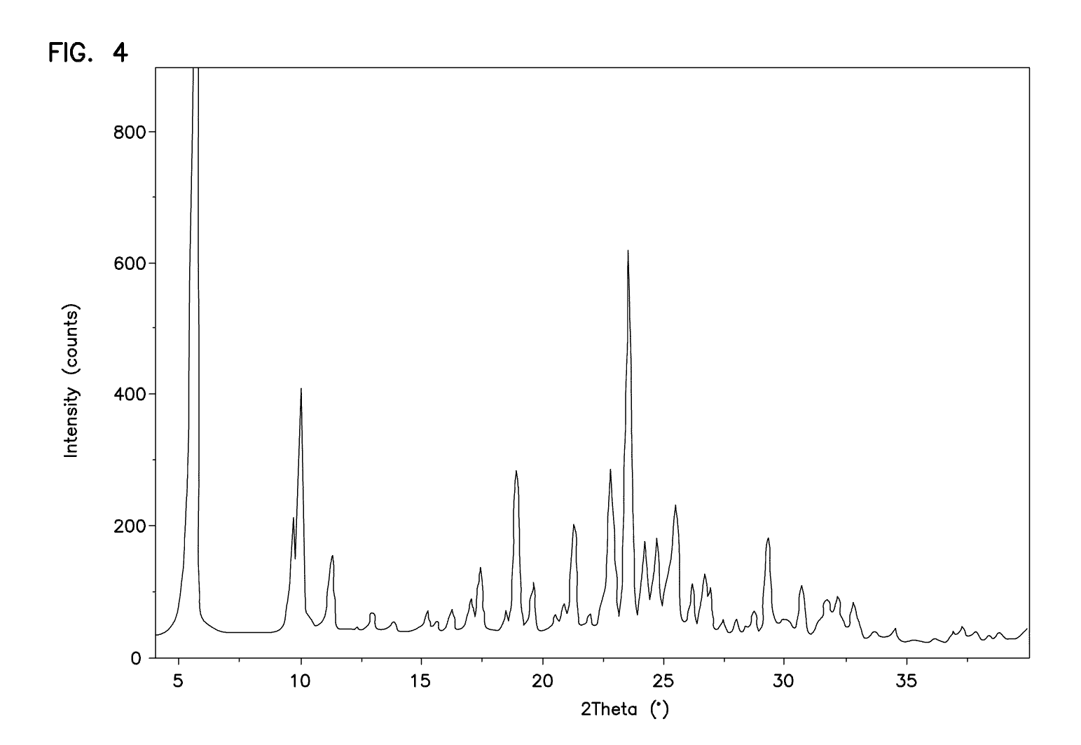

The present invention further relates to process for preparing crystalline Form of Erlotinib HCl characterized by data selected from the group consisting of: a powder XRD pattern having peaks at about 10.1 and 17.4±0.2 degrees 2-theta and any 3 peaks selected from the list consisting of: 5.7, 10.1, 17.4, 18.9, 21.3, 23.6 and 29.3±0.2 degrees 2-theta, a PXRD pattern described on FIG. 4, and combination thereof.

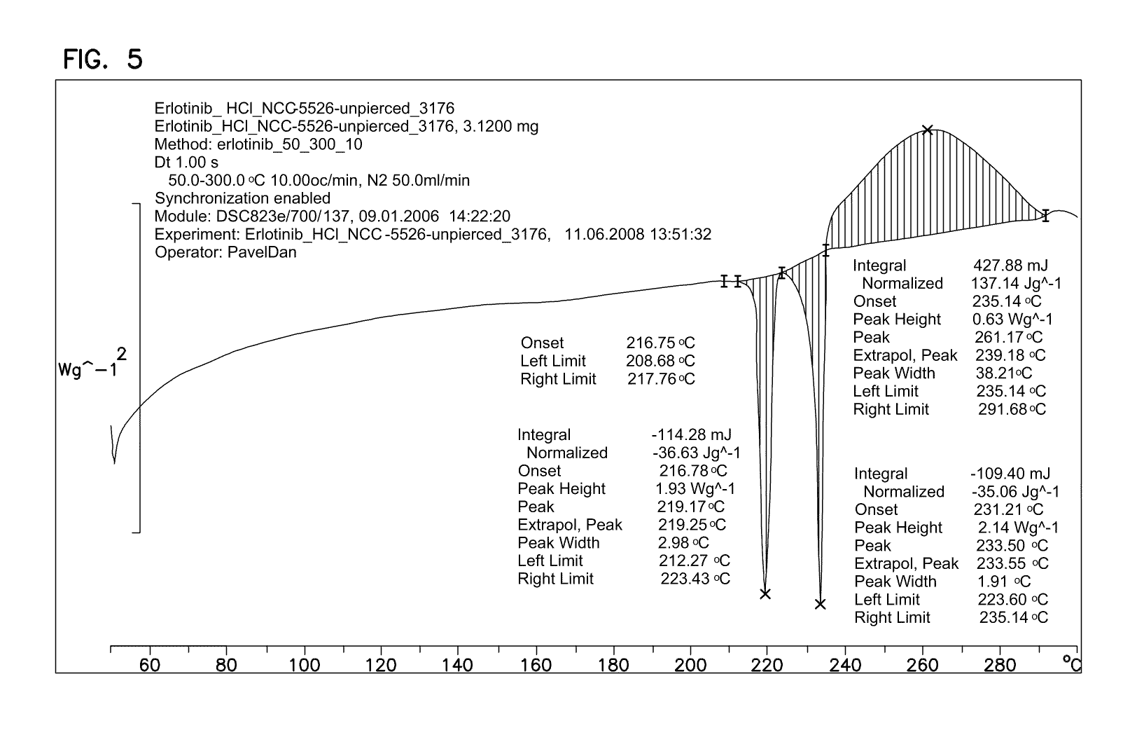

This crystalline form can be further characterized by data selected from the group consisting of: a DSC endothermic peak at about 219° C. and 234° C., a thermogram depicted in FIG. 5, a DSC onset temperature of about 217° C., and combination thereof.

The said crystalline form of erlotinib HCl is also characterized by a content of no more than about 20% by weight of other crystalline forms of erlotinib HCl, preferably not more than 10% by weight, more preferably not more than 5% by weight of other crystalline forms of erlotinib HCl. Preferably potential contamination e.g. by Erlotinib hydrochloride form B provided by % by weight is measured by PXRD or by C-13 solid state NMR. When measured by PXRD, the content is determined by using one or more peaks selected from the following list of peaks 6.3, 7.8, 12.5, 13.4 and 20.2±0.2 degrees 2-theta. More preferably XRD diffraction peak at about 6.3±0.2 degrees 2-theta. For quantification of Form B in Form A especially small percentages of Form B in Form A, the general chapter on “Characterization of crystalline solids by XRPD” of the European Pharmacopoeia 5.08, chapter 2.9.33 may be followed.

The said process comprises:

a) concentrating a first mixture comprising CMEQ having the following formula:

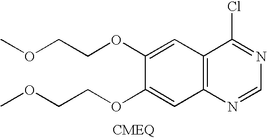

3-ethynylbenzamine (“3-EBA”) having the following formula:

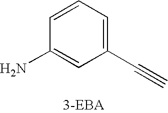

and 2-butanone;

b) adding 3-ethynylbenzamine (“3-EBA”) and an amount of water to obtain a second mixture; and

c) heating the second mixture to obtain a suspension comprising the said crystalline form of Erlotinib HCl.

The first mixture comprising CMEQ, 3-EBA and 2-butanone is prepared by a process comprising reacting 6,7-bis(2-methoxyethoxy)quinazolinone (“MEQO”) having the following formula:

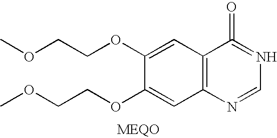

and thionyl chloride in a mixture of dichloromethane and catalyst to obtain a solution comprising CMEQ and dichloromethane, adding 3-EBA to the said solution to obtain the said first mixture.

The reaction of MEQO and thionyl chloride in a mixture of dichloromethane and catalyst is done by suspending MEQO in a mixture of dichloromethane and the catalyst and adding thionyl chloride to the suspension. Preferably, the addition of thionyl chloride provides a solution, which transforms into a suspension in a period of about 2 minutes to about 10 minutes.

Preferably, the catalyst is dimethylformamid (“DMF”).

Preferably, the reaction of MEQO and thionyl chloride further comprises heating the said suspension to obtain a solution. Preferably, heating is to at least about reflux temperature. Preferably, the heating is done for a period of about 15 hours, during which the progress of the reaction is monitored by HPLC. The progress of the reaction can be determined by measuring the amount of the residual starting material, 6,7-bis(2-methoxyethoxy)quinazolinone (“MEQO”), preferably, by HPLC.

Ordinarily, the reaction of MEQO and thionyl chloride further comprises a work-up process, prior to the addition of 3-EBA. Preferably, the work-up process comprises cooling the said solution; adding water and a base to the solution providing a two-phase system; separating the phases; and washing the organic phase with water.

Preferably, the base added is an inorganic base or an organic base. Preferably the inorganic base is Na2CO3 or NaHCO3. Preferably, the organic base is triethylamine. Most preferably the base added is sodium hydroxide. Preferably, the said solution is basified by the addition of the base to a pH of about 7.5 to about 8.0.

Preferably, the washed organic phase is the solution to which 3-EBA is added, thus providing a mixture, and this mixture is concentrated leading to a first residue.

Typically, the concentration of the above mixture is done to remove residual dichloromethane. This first reside is then combined with 2-butanone obtaining the first mixture, which is concentrated again. The concentration preferably yields a concentrate that still may comprise residual dichloromethane, for example less than 2% by weight. Further, the obtained concentrate is then combined with 3-EBA and water yielding the second mixture.

Further, the second mixture in step c) is preferably heated to ensure that the formation of Erlotinib HCl is completed. Preferably, Erlotinib HCl is formed as a precipitate. Preferably, heating is to about 20° C. to about reflux, more preferably to about 50° C. to about reflux temperature. Preferably, heating is for a period of about 1 hour to about 12 hours, more preferably about 3 hours to about 7 hours. Most preferably, heating is for a period of about 5 hours.

The precipitated crystalline Erlotinib HCl can be recovered from the suspension. The recovery can be done, for example by cooling the suspension, filtering the crystalline, washing the filtered crystalline, and drying. Preferably, cooling is to a temperature of about room temperature. Preferably, drying is to a temperature of about 30° C. to about 90° C., more preferably the temperature is about 50° C. to about 70° C. Most preferably drying is to a temperature of about 60° C.

The above crystalline forms of Erlotinib HCl (Al and the other one) can be used to prepare a pharmaceutical composition.

The present invention further encompasses 1) a pharmaceutical composition comprising any one, or combination, of crystalline Forms of Erlotinib HCl and at least one pharmaceutically acceptable excipient and 2) the use of any one, or combination, of the above-described crystalline Forms of Erlotinib HCl, in the manufacture of a pharmaceutical composition, wherein the pharmaceutical composition can be useful for the treatment of patients with locally advanced or metastatic non-small cell lung cancer (NSCLC) after failure of at least one prior chemotherapy regimen.

The pharmaceutical composition of the present invention can be in a solid or a non-solid form. If the pharmaceutical composition is in a non-solid form, any one, or combination, of the crystalline Forms Erlotinib HCl within the composition, are retained as solid(s) in the non-solid pharmaceutical composition, e.g., as a suspension, foam, ointment and etc.

The pharmaceutical composition can be prepared by a process comprising combining any one, or combination, of the above-described crystalline Forms Erlotinib HCl with at least one pharmaceutically acceptable excipient. The crystalline Forms Erlotinib HCl form can be obtained by any of the processes of the present invention as described above.

The pharmaceutical composition can be used to make appropriate dosage forms such as tablets, powders, capsules, suppositories, sachets, troches and losenges.

Any one, or combination, of the above-described crystalline Forms Erlotinib HCl of the present invention, particularly in a pharmaceutical composition and dosage form, can be used to treat patients with locally advanced or metastatic non-small cell lung cancer (NSCLC) after failure of at least one prior chemotherapy regimen comprising administering a treatment effective amount of the one, or combination, of the crystalline Forms Erlotinib HCl in the patient. The treatment effective amount or proper dosage to be used can be determined by one of ordinary skill in the art, which can depend on the method of administration, the bioavailability, the age, sex, symptoms and health condition of the patient, and the severity of the disease to be treated, etc.

### EXAMPLES

### PXRD

X-ray powder diffraction (XRPD) was performed on X-Ray powder diffractometer: PanAlytical X'pert Pro powder diffractometer, CuKα radiation, λ=1.541874 Å. X'Celerator detector active length (2 theta)=2.122 mm, laboratory temperature 22-25° C. Zero background sample-holders. Prior to analysis, the samples were gently ground by means of mortar and pestle in order to obtain a fine powder. The ground sample was adjusted into a cavity of the sample holder and the surface of the sample was smoothed by means of a microscopic glass slide.

### Single-Crystal XRD Analysis

Data were collected on Xcalibur PX, Cu Kα (wavelength=1.540598 angstroms) using combined φ and ω scans at 200K. All non-hydrogen atoms were refined anisotropically; hydrogen atoms were refined riding in expected geometric positions. Data collection: CrysAlis RED; cell refinement: CrysAlis RED; data reduction: CrysAlis RED; program used to solve structure: SIR92 (Altomare et al., 1994); program used to refine structure: CRYSTALS; Data export (Appendix 1) was done by Platon.

### DSC

DSC measurements were performed on Differential Scanning Calorimeter DSC823e (Mettler Toledo). Aluminum crucibles 40 μl with lid were used for sample preparation. The lid was not perforated before analysis. Typical weight of sample was 1-4 mg. Program: temperature range 50° C.-300° C., 10° C./min under flow of nitrogen 50 ml/min.

Onset temperature is determined as a crossing of tangents constructed on the baseline and at start of the event peak.

### Example 1

### Preparation of Erlotinib Hydrochloride Form AL

Erlotinib base (50 mg) was dissolved in methylethylketone (MEK, 10 ml) by slight heating at 50° C. and allowed to cool to 20° C. The glass bottle with the erlotinib base solution was placed into a closed glass container (500 ml volume) and diluted hydrochloric acid (300 μl of 35% HCl and 500 μl of water) was dripped to the bottom of container. Slow diffusion of HCl vapors within 3 days facilitated slow crystallization of erlotinib hydrochloride. Crystals of erlotinib hydrochloride were separated by filtration, washed with t-butyl methyl ether (TBME, 10 ml) and dried on air.

### Example 2

### Preparation of Crystalline Form of Erlotinib HCl Characterized by Data Selected from the Group Consisting of: a Powder XRD Pattern having Peaks at about 10.1 and 17.4±0.2 Degrees 2-Theta and any 3 Peaks Selected from the List Consisting of: 5.7, 10.1, 17.4, 18.9, 21.3, 23.6 and 29.3±0.2 Degrees 2-Theta, a PXRD Pattern Described in FIG. 4, and Combinations Thereof

6,7-Bis(2-methoxyethoxy)-4-quinazolinone (10 g; 0.034 mol) was suspended in CH₂Cl₂ (173 g) and DMF (2 g). Thionyl chloride (7 g; 0.059 mol) was added and a yellow and clear solution was obtained. After about 10 min., a precipitation occurred. The mixture was heated to reflux for 15 hours (after 5 hours a solution was obtained) until residual 6,7-bis(2-methoxyethoxy)-4-quinazolinone <0.3% (by HPLC). The mixture (yellowish solution) was cooled to 15° C. and H₂O (50 mL) was added. The mixture pH is adjusted to 7.5-8.0 by addition of 30% NaOH (about 11.5 g) under vigorous stirring. After separation of the phases, the organic layer was washed with H₂O (50 mL). 3-Ethynylbenzamine (4.4 g) was added to the organic phase and the mixture was concentrated by distillation to a total weight of about 30 g. 2-Butanone (10 g) was added to the residue and the mixture was heated to reflux. Residual dichloromethane was removed by distillation and the reaction mixture was refluxed for 20 hours. Additional 3-ethynylbenzamine (0.4 g) and water (2 g) were added and reaction mixture was refluxed for 5 additional hours until complete reaction (by HPLC: 6,7-bis(2-methoxyethoxy)-4-quinazolinone about 1%).

The suspension was cooled to room temperature, stirred for 1 hour, filtered and the solid washed with butanone (20 g). The wet solid was dried overnight under vacuum at 60° C. 14.4 g (97% yield) of Erlotinib hydrochloride were obtained.

### Example 3

### Preparation of Crystalline Erlotinib Base Form G2

6,7-Bis-(2-methoxyethoxy)-4(3H)-quinazolinone (“MEQO”) (10 g; 0.034 mol) was suspended in CH₂Cl₂ (130 mL) and DMF (2 mL). Thionyl chloride (7 g; 0.059 mol) was added and a yellow and clear solution is obtained. After about 10 min the starting material precipitated again. The mixture was heated to reflux for at least 8 h (after about 5 h a solution was obtained) until residual MEQO<0.3% (In Process Control 1). The mixture (yellowish solution) was cooled to 15° C. and H₂O (50 mL) was added (exothermic quench of residual thionyl chloride). The mixture pH was adjusted to 7.5-8.0 by addition of 30% NaOH (about 11.5 g) under vigorous stirring. After separation of the phases, the organic layer was washed with H₂O (50 mL). The organic phase was concentrated under vacuum to a total volume of about 30-40 mL. The mixture was diluted with i-PrOH (isopropyl alcohol; 150 mL) and the mixture was concentrated until about 5 volumes of solvent were removed (In Process Control 2: residual CH₂Cl₂<2%, by vol.). The mixture was heated at 40° C. and 3-EBA (4.4 g; 0.038 mol) was added. The mixture was additionally diluted with i-PrOH (75 mL) in order to obtain a stirrable suspension and it was stirred at 40° C. for 8 h (In Process Control 3: residual 4-chloro-6,7-bis(2-methoxyethoxy)quinazoline (“CMEQ”)<2%). At this stage the mixture already contains Erlotinib HCl. The reaction mixture was cooled to 20-25° C. and AcONa (2.8 g; 0.034 mol) was added. After two hours stirring, the suspension was filtered and the solid was washed with i-PrOH (25 mL). The wet solid was dried under vacuum at 45-50° C. for 3 h to give ERL-Base.

### Example 4

### Conversion of Erlotinib Base Form G2 to Erlotinib Hydrochloride

ERL-Base form G2 obtained from ex. 3 (11.5 g, corresponding to 0.025 mol and to 10 g 100% assay) was suspended in methylisobutylketone (“MIBK”) (200 mL) and H₂O (50 mL), the resulting mixture was heated at 65-70° C. until a clear solution was obtained. The phases were separated and the organic layer was additionally three times washed with H₂O (3×50 mL) at 65-70°. The organic phase was concentrated until about 6 volumes of solvent were removed and the starting mixture volume was restored by addition of fresh MIBK. In Process Control: Karl Fisher≦0.4%. The mixture was heated to 55-60° C. under stirring (about 300 RPM) and 32-37% HCl (2.8 g; 0.028 mol was added causing the immediate precipitation of the hydrochloride salt. The mixture was cooled to 20-25° C. in about 1 h, and then it was kept at the same temperature for 1 h. The suspension was filtered and the solid was washed with i-PrOH (5 mL). The wet solid was dried under vacuum at 45-50° C. for 15-18 h to give ERL-HCl as a white solid (10.4 g; 0.024 mol). The yield was 95%.

### Comparative Example 5

### Attempt to Prepare Erlotinib Base in the Presence of AcOK (Potassium Acetate)

MEQO 6,7-Bis-(2-methoxyethoxy)-4(3H)-quinazolinone (10 g; 0.034 mol) was suspended in CH₂Cl₂ (130 mL) and DMF (2 mL). Thionyl chloride (7 g; 0.059 mol) was added and a yellow and clear solution is obtained. After about 10 min the starting material precipitated again. The mixture was heated to reflux for at least 8 h (after about 5 h a solution was obtained) until residual MEQO<0.3% (In Process Control 1.) The mixture (yellowish solution) was cooled to 15° C. and H₂O (50 mL) was added (exothermic quench of residual thionyl chloride). The mixture pH was adjusted to 7.5-8.0 by addition of 30% NaOH (about 11.5 g) under vigorous stirring. After separation of the phases, the organic layer was washed with H₂O (50 mL). The organic phase was concentrated under vacuum to a total volume of about 30-40 mL. The mixture was diluted with i-PrOH (150 mL) and the mixture was concentrated until about 5 volumes of solvent were removed (In Process Control 2: residual CH₂Cl₂<2%, by vol.). The mixture was heated at 40° C. and 3-EBA (4.4 g; 0.038 mol) was added. The mixture was additionally diluted with i-PrOH (75 mL) in order to obtain a stirrable suspension and it was stirred at 40° C. for 8 h (In Process Control 3: residual CMEQ 4-chloro-6,7-bis(2-methoxyethoxy)quinazoline <2%). The reaction mixture was cooled to 20-25° C. and AcOK (3.3 g; 0.034 mol) was added. After two hours stirring, the suspension was filtered and the solid was washed with i-PrOH (25 mL). The wet solid was dried under vacuum at 45-50° C. for 3 h to give ERL-hydrochloride.

1. A process for preparing crystalline form of Erlotinib base form G2 characterized by data selected from the group consisting of: an X-ray powder diffraction pattern with peaks at about 6.5, 12.9, 17.3, 18.3 and 22.4 degrees two-theta±0.2 degrees two-theta, and a PXRD pattern as depicted in FIG. 7, comprising:
reacting sodium acetate and erlotinib hydrochloride in an alcohol to obtain a precipitate containing crystalline Erlotinib base form G2.

2. The process of claim 1, wherein sodium acetate is added to a reaction mixture comprising erlotinib hydrochloride and the alcohol.

3. The process of claim 2, wherein the alcohol is isopropyl alcohol.

4. The process of claim 1, wherein the alcohol is isopropyl alcohol.

5. The process of claim 1, wherein the precipitate contains solid NaCl.

6. A process for preparing an Erlotinib salt comprising preparing crystalline Erlotinib base form G2 according to the process of claim 1, and converting it to an Erlotinib salt.

7. The process of claim 6, wherein the salt is hydrochloride salt.

8. The process of claim 6, further comprising separating NaCl from crystalline Erlotinib form G2 prior to the converting step.

9. The process of claim 8, further comprising
suspending the precipitate in water immiscible solvent and water;
inducing separation into at least an aqueous phase and an organic phase containing erlotinib base; and
acidifying the organic phase to yield the erlotinib salt.

10. The process of claim 9, wherein the water immiscible solvent is a water immiscible ketone.

11. The process of claim 10, wherein the water immiscible ketone is methyisobutylketone.
